(19) United States
(12) Patent Application Publication
(10) Pub. No.: US 2009/0110739 A1
(43) Pub. Date: Apr. 30, 2009

(54) Targeted cancer chemotherapy using synthetic nanoparticles

(75) Inventors: Andras G. Lacko, Fort Worth, TX (US); Walter J. McConathy, Midland, TX (US)

Correspondence Address: JACKSON WALKER LLP, 901 MAIN STREET, SUITE 6000, DALLAS, TX, 75202-3797, US

(73) Assignee: University of North Texas Health Science Center at Forth Worth, Forth Worth, TX (US)

(21) Appl. No.: 12/152,559

(22) Filed: May 15, 2008

Related U.S. Application Data
(60) Provisional application No. 60/930,439, filed on May 15, 2007.

(51) Int. Cl. A61K 9/14; A61K 31/337; A61K 31/35; A61K 39/395

(52) U.S. Cl. 424/491; 514/449; 514/459; 424/181.1

(57) ABSTRACT

Compositions and methods for delivery of a pharmaceutical to an individual. Delivery vehicles are provided in a formulation of a pharmaceutical that is encapsulated in a synthetic self assembled nanoparticle that includes a lipid binding protein and a lipid monolayer. The interior of the particle represents a hydrophobic core region where lipophilic highly-water insoluble drug molecules may be incorporated. In contrast to liposomes, that include an aqueous interior core surrounded by phospholipid bilayer, the drug carrier nanoparticle described here is composed of a monolayer and a hydrophobic interior.

### RELATED APPLICATIONS

The present application claims priority to U.S. Provisional Patent Application Ser. No. 60/930,439. filed May 15, 2007, the entire content of which is incorporated herein by reference.

### STATEMENT OF RIGHTS TO INVENTIONS MADE UNDER FEDERALLY SPONSORED RESEARCH

The early phases of the work leading to this invention were made in part during work supported by grant DAMD170110582 form the Department of Defense Congressionally Directed medical research Programs (USA-CDMRP/Breast Cancer Concept Award). The government may have certain rights in the invention.

### BACKGROUND

This invention pertains to methods and compositions that can be used in drug delivery, particularly as agents for cancer chemotherapy. Specifically, the invention pertains to synthetic self-assembled nanoparticles that include a lipid binding apolipoprotein, three lipids and a drug, forming a spherical-core containing structure, resembling high density lipoproteins (HDL). Nanoparticles are particles with a diameter of less than about 1000 nm.

Most cancer chemotherapeutic agents cannot be administered by themselves as pure chemicals but have to be included in bio-compatible formulations that enhance solubility, increase circulatory residence time of the therapeutic agent, and minimize the undesirable side effects and to alleviate drug resistance.

Numerous approaches have been developed to overcome these difficulties, including solid lipid particles, emulsions, and liposomes etc., however, the delivery of the poorly water soluble (hydrophobic) pharmaceuticals remains especially problematic as most of the body compartments, including the blood circulation and intracellular fluids, represent an aqueous environment. As a result, the direct injection of hydrophobic therapeutic agents often results in harmful side effects due to hypersensitivity, hemolysis, cardiac and neurological symptoms. Consequently, there is need for more effective formulations of hydrophobic drugs to improve their biocompatibility and therapeutic efficiency.

### I Plasma Lipoproteins

Rapid advances in drug discovery and development have also spawned numerous innovative drug delivery approaches. Although these efforts have primarily focused on optimizing the performance of drugs targeted for the current market, enhanced delivery of drugs may also result in the revitalization of marginally effective or failed formulations due to their original poor solubility. Plasma lipoproteins have long been considered as appropriate models for drug delivery vehicles, particularly because of their potential for transporting chemicals with low water solubility. Additional features that render lipoproteins particularly suitable for drug delivery are their natural, bio-compatible components, their small size, the ability to deliver the drug itself rather than a prodrug or conjugate and the receptor mediated their uptake or the uptake of their payload, particularly by cancer cells. There has been increased activity in the patenting of lipoprotein type formulations in the last several years, primarily with the aim of developing enhanced drug delivery vehicles.

### II General Properties of Plasma Lipoproteins

Plasma lipoproteins are made up of protein and lipid components to form a globular complex, designed to transport water insoluble lipids in a water based physiological environment. The two phase structure of lipoproteins includes an outer shell made up of detergent like (amphiphilic) phospholipid monolayer and protein components and an interior core containing highly hydrophobic lipids (triacylglycerols and cholesteryl esters). This two phase configuration allows lipoproteins to fulfill their roles as drug delivery agents, particularly in the transport of water insoluble drugs.

TABLE 1
Composition and basic properties of human plasma Lipoproteins.

| | | | | | | | Primary |
|---|---|---|---|---|---|---|---|
| | | | Density | | | | Protein |
| Lipoprotein | Size | | (g/mL) | Half-life | | Function | Component |
| Chylomicron | 75-1200 | nm | <0.960 | 5-20 | min. | Transport | Apo-AI, -AIV¹ |
| | | | | | | lipid from | |
| | | | | | | gut to liver | |
| VLDL² | 30-80 | nm | 0.96-1.006 | 5 | min | Endogenous | ApoB-100³ |
| | | | | | | lipid | |
| | | | | | | transport | |
| LDL⁴ | 18-25 | nm | 1.019-1.063 | 1-2 | days | Endogenous | ApoB-100⁵ |
| | | | | | | lipid | |
| | | | | | | transport | |
| HDL⁶ | 8-14 | nm | 1.063-1.210 | 3-5 | days | Cholesterol | ApoA-I⁷ |
| | | | | | | transport | |

¹“apolipoprotein AI, apolipoprotein AIV”
²Very Low Density Lipoprotein
³“apolipoprotein B-100”
⁴Low Density Lipoprotein
⁵“apolipoprotein B-100”
⁶High Density Lipoprotein
⁷“apolipoprotein AI”

The formulation approach made possible by lipoprotein-based drug delivery systems may be favorable over alternative strategies, such as the development of water soluble prodrugs, by its applicability to a variety of poorly water soluble drugs with no or minor modification of the technology, the possibility of organ- or tissue-selective targeting, the projected relative ease of regulatory approval and relatively modest cost.

### III Low Density Lipoprotein (LDL) Based Formulations

PCT Patent Publication No. WO/1998/046275 by Counsell and Pohland describes a blood-pool carrier for lipophilic imaging agents.

PCT Patent Publication No. WO/1986/07539 by Masquelier et al. describes a method for the production of a macromolecular carrier loaded with a biologically active substance.

PCT Patent Publication No. WO/1998/013385 by Halbert et al. describes non-naturally occurring lipoprotein particles.

U.S. Patent Publication 2003/0008014 by Shelness describes truncated apolipoprotein B-containing lipoprotein particles for delivery of compounds to tissues or cells.

U.S. Patent Publication 2004/0204354 by Nelson et al. describes artificial low-density lipoprotein carriers for transport of substances across the blood-brain barrier.

U.S. Pat. No. 6,117,454 to Kreuter, et al. describes drug targeting to the nervous system by nanoparticles.

PCT Patent Publication No. WO/2006/073419 by Glickson et al. describes lipoprotein nanoplatforms.

### IV High Density Lipoprotein (HDL) Based Formulations

There are some difficulties regarding the existing nomenclature regarding of what reconstituted HDL (rHDL) actually represents. In fact, the terms “reconstituted” and “recombinant” have both been used to describe synthetic HDL-type particles that represent both discoidal and spherical nanoparticles as “rHDL”. These issues are important as the respective drug carrying capacity and stability of the nanoparticles depend a great deal on their spherical vs discoidal configuration.

U.S. Pat. No. 7,053,049 to Luescher et al. describes a method for treating unstable angina pectoris.

U.S. Pat. No. 6,998,388 to Cockerill et al. describes high density lipoprotein against organ dysfunction following hemorrhagic shock.

U.S. Pat. No. 6,514,523 to Sparks describes carrier particles for drug delivery and process for preparation.

U.S. Patent Publication 2004/0229794 to Ryan and Oda describes a lipophilic drug delivery vehicle and methods of use thereof.

U.S. Pat. No. 5,652,339 to Lerch, et al. describes a method of producing reconstituted lipoproteins.

U.S. Pat. No. 5,128,318 to Levine, et al. describes reconstituted HDL particles and uses thereof.

U.S. Pat. No. 5,089,602 to Isliker, et al. describes a process for the manufacture of apolipoproteins from human blood plasma or serum.

### V Conclusions

Despite these ideas, there are no lipoprotein based formulations currently in use in the pharmaceutical industry. Recent studies on Abraxane and serum albumin point to the importance of advanced delivery vehicles (including lipoproteins) in intravenous therapy. Considering that their effective drug delivery capabilities, including the potential for precise targeting, the continued development of lipoprotein based formulations should provide robust drug delivery platforms and thus effective therapeutic tools.

Macromolecular complexes containing lipids (liposomes) have been utilized as delivery vehicles for chemotherapeutic agents. However, intact lipoproteins, specifically, high density lipoprotein (HDL) type lipid/protein complexes, have not been used for this purpose. Specificity of bioactive agent delivery is an area in cancer treatment that continues to need improvement. Drug exposure to normal cells renders current chemotherapeutic treatment therapies relatively toxic to normal, non diseased cells in an animal being treated. The overall toxicity of using these drugs in human cases results in undesirable side effects to the patient that even further reduce the patient's quality of life. Increased specificity is of critical importance in cancer chemotherapy where the relative toxicity of highly toxic chemotherapeutic agents to non-cancerous cells is a problem.

The primary course of initial clinical management of cancer is surgery. However, patients benefit significantly from adjuvant chemotherapy. A significant portion of cancer cases present at advanced cancer stages at diagnosis. Hence, chemotherapy following surgery is generally necessary. To enhance the efficacy of chemotherapy, multiple agents, longer and more intensive treatment, and combination surgery and chemotherapy clinical regimes have been utilized. These approaches have in some cases resulted in improved survival and cure rates. Currently, a number of therapeutic agent combinations, including cisplatin and taxol, are used. Doxorubicin (DOX) is also used, and has been reported to extend patient survival and 10 suppression of drug resistance, particularly in the treatment of breast and ovarian cancers.

Recent clinical trials with several chemotherapeutic agents report increasing survival and cure rates. Despite these reports, the toxic side effects of these agents continue to be of major concern. Consequently, improvement of delivery vehicles for cancer chemotherapy is a primary goal for enhancing its effectiveness.

In order to overcome or reduce the toxic side effects of chemotherapeutic agents, liposomes and other delivery vehicles have been investigated. The potential of liposomes as drug delivery agents has long been recognized. Liposomes have been used routinely in the delivery of proteins, interleukins, cancer chemotherapeutic agents and antisense oligonucleotides. The next generation of liposomes involves new approaches to improve the efficiency of drug delivery. These modifications include the use of specialized lipids or polyethylene glycol as liposome components for extending the residence times of the particles in the circulation and the attachment of targeting signals such as glycolipids, proteins antigens or antibodies to the liposome complex. Despite these improvements and advances, toxic side effects remain a serious concern, particularly during the delivery of highly effective drugs during cancer chemotherapy.

Earlier studies have shown that the efficacy of liposome drug delivery was inversely related to the diameter of the liposome particle. The average HDL particle has a diameter of 100-200 A. Hence, even the smallest liposomes have a diameter five times larger than that of the average HDL particle.

### SUMMARY

The present invention overcomes those problems in the prior art associated with drug delivery and generalized toxicity by providing a uniquely formulated packaging and targeting preparation that has particular application in the delivery of anticancer agents. The present invention discloses the use of HDL type lipid/protein complexes as delivery vehicles for cancer chemotherapeutic agents and other pharmaceutical agents. By way of example, the present invention provides for the incorporation of antitumor agents into HDL-type particles. In addition, a method by which HDL type lipid/protein complexes containing hydrophobic pharmaceutical agents are generated is provided that represents a self assembly process, possibly facilitated by the added cholates, of the specific constituents of HDL together with the desired drug. In some embodiments, phosphatidylcholine, cholesterol, cholesteryl oleate, drug and apolipoprotein AI (apoA-I) are used in the formula. The HDL-drug particles created may be defined as having the approximate size of HDL, based on analyses by preparative ultracentrifugation and gel chromatography. A chemotherapeutic drug that may be incorporated in the HDL complex during sonication by way of example, is doxorubicin (DOX), doxorubicin derivatives (examples are epirubicin idarubicin and zyn-linked doxorubicin), paclitaxel (PTX), as well as combinations of these agents.

The present invention in one aspect provides a novel delivery system for anticancer agents to treat malignant tumors using HDL-type particles, as transport vehicles. The use of HDL over liposomes and other artificial complexes as transport vehicles is advantageous because they are smaller in size and their contents are rapidly internalized by receptors of specific cells, including receptors on the surface of tumor tissue. An additional advantage of HDL as a delivery vehicle for chemotherapeutic agents lies in the fact that the uptake of HDL core components by cells is facilitated by specific cell surface receptors. Recent studies indicate that the proliferation of adenocarcinoma cells and breast cancer cells is dependent on the uptake of HDL and HDL components. Although liposomes have been utilized as anti-cancer drug delivery vehicles, the use of HDL in this particular manner is novel and unique.

The present invention further provides a method for improving the delivery of anti-cancer drugs to malignant tumors. Other therapeutic compounds requiring a delivery vehicle may also be included in the formulation. It is expected that, with the enhanced drug delivery specificity provided in the formulation significant improvement of the prognosis for cancer survivors will be achieved. The HDL transport vehicle is also expected to decrease the relative toxicity of chemotherapeutic agents. Additional advantages of the invention exist in part on the flexibility of the HDL structure of the formulation. For example, the structure of HDL may be changed to enhance drug delivery specificity. This may be accomplished by using different variations and combinations of specific lipids and protein constituents. In addition, covalent attachment of ligands, and the use of synthetic peptides substituting for apolipoproteins, may be employed to even further enhance the tumor cell delivery specificity of the molecule. Further variation in compositional properties of the lipids can readily be achieved by introducing phosphoglycerides with a desired composition or employing other lipids (e.g. sphingomyelin, cationic lipids) when preparing the HDL-lipid mix. Alteration of surface properties by chemical modification of lipids or apolipoproteins may also be used to alter the specificity of tissue delivery and to enhance the effectiveness of therapies designed for targeting specific metastatic tumors. Because circulating HDL contains apolipoproteins (A-11, A-IV, C-1, C-11, C-111, E and F), other than apoAI, additional of these alone or in combination may be used to enhance specificity of delivery to certain types of metastatic tumors. Peptide analogs of these apolipoproteins may also be employed in the design of specific HDL preparations as described for apoAI.

While a number of tissues express HDL receptors (including the liver, adrenals and ovaries), rapidly growing cells (such as tumor cells) are more likely to acquire hydrophobic core components from HDL through HDL receptors, as compared to quiescent cells. The present invention offers a preferential impact on tumor cells versus normal cells by the utilization of the HDL/protein complex delivery system by employing this phenomenon in the design of the formulation delivery system. By utilizing the HDL transport vehicle in the formulation, the specificity of drug uptake by targeted tumor cells is increased.

The term “complex” as is used in describing the present invention, is defined as a composition composed primarily of a phospholipid, cholesterol and a polypeptide. A polypeptide is defined as an apolipoprotein AI or an apolipoprotein AI-like polypeptide. The phospholipid, cholesterol and polypeptide are held together in the complex by non-covalent bonds. The complex is relatively stable, and is thus distinct from chemical mixtures and other such formulations, The formulations are to be prepared in a pharmaceutically acceptable carrier, and are suitable as pharmaceutical treatments. The stability of the present complex molecules is demonstrated in the preparative ultracentrifugation and gel chromatography tests using the preparations. These examinations of the product indicate the presence of a stable macromolecular entity. The complex of the present invention has also been found to be stable when subjected to dialysis. One of the several advantages of the present invention relates to the reduced toxicity of the preparation. This advantage is particularly important in preparations that include anti-cancer drugs. The macromolecular complex of the invention may be further described as resembling high density lipoproteins (HDL), in terms of the chemical composition and molecular dimensions of the complex. The molecular weight of the complex is estimated to be approximately 100,000-200,000 Daltons.

The resemblance of the complex to HDL is associated with the large protein component of the molecule (−70% of total mass), and from the protein of the complex, which is primarily apolipoprotein AI. Apolipoprotein AI is the largest protein component of circulating (normal) HDL.

In some embodiments, the complex of the invention may be modified to enhance its performance by alteration of its lipid composition. By way of example, chemical modifications of the apolipoprotein and lipid components may be made by inclusion of apolipoproteins other than apolipoprotein AI. These proposed changes would not alter the basic physical/chemical characteristics of the complex.

Examples of other chemotherapeutic/cytotoxic/imaging agents that could be incorporated into HDL and use the consequent modified HDL as a delivery vehicle to malignant cells. A number of these agents would require chemical modification or covalent attachment to HDL components for incorporation into the HDL particle.

TABLE 2

| | |
|---|---|
| ANTIBIOTIC AGENTS | |
| Bleomycin | DactinoLnycin |
| Daunorabicin | Mitomycin |
| Mitomycin | Mitoxantrone, |
| Plicamycin | |
| ALKYLATING AGENTS | |
| Busulfan | Carboplatin |
| Carmustin | Chlorambucil |
| Cisplatin | Cyclophosphamide |
| Dacarbazine | Ifosfamide |
| Lomustine | Mechlorethamine |
| Melphatan | Semustine, |
| Thiotepa | |
| ANTI-METABOLITE AGENTS | |
| Qqarabine, | Fluorouracil |
| Floxuridine | Methotrexate |
| Mercaptopurine | |
| MITOTIC INHIBITOR AGENTS | |
| Etoposide | Teniposide |
| Vinblastine | Vincristine, |
| Vindesine, | |
| IMAGING AGENTS | |
| Gandolinium - EDTA | I-131 labeled lipids |
| IHDA, 15-(p-I-phenyl)pentadecanoic | |
| acid (IPPA) | |
| I-131 fatty acids | |
| OTHER AGENTS | |
| L-Asparaginase | Hydroxyurea |
| Procarbazine | |

Following long-standing patent law convention, the terms “a” and “an” mean “one or more” when used in this application, including the claims.

The following Table 3 lists examples of various cell lines that may be used in screening particular HDL-drug preparations for use in treatment of a specific type of cancer.

TABLE 3
Representative Cell Lines

| | ATCC | | | |
|---|---|---|---|---|
| Cell Line | Number | Source | Type | Resistance |
| A2780 | N/A | Ovarian | | Sensitive |
| A2780-R | | Ovarian | | Resistant |
| Caov-3 | HTB-75 | Ovarian | Adenocarcinoma | |
| Caov-4 | HTB-76 | Ovarian | Adenocarcinoma. | |
| | | Metastatic site: | | |
| | | Subserrosa of the | | |
| | | fallopian Tube | | |
| Hs 904.T | CRL-7651 | Ovarian | | |
| LNCap.FGC | CRL-1740 | Prostate | Carcinoma | |
| | | Metastatic site: | | |
| | | Left supraclavicular | | |
| | | lymph node | | |
| MDAH 2774 | CRL-1030 | Ovarian | Adenocarcinoma | |
| | | Papillary | | |
| | | cystadenocarcinoma | | |
| OV-1063 | CRL-2183 | Ovarian | Adenocarcinoma | |
| OVCAR-3 | HTB-161 | Ovarian | Adenocarcinoma | Resistant to |
| | | | | DOX and |
| | | | | cisplatin |
| P388 | | CHO | | Sensitive and |
| | | | | resistant sublines |
| PA-1 | CRL-1572 | Ovarian | Teratocarcinoma | |
| | | Metastatic site: | | |
| | | Ascites | | |
| SK-OV-3 | HTB-77 | Ovarian | Adenocarcinoma, | Resistant to |
| | | Metastatic site: | | DOX and |
| | | Ascites | | cisplatin |
| SW626 | HTB-78 | Ovarian | Papillary cysta | |
| | | | Adenocarcinoma | |
| UCI 101 | UC Orange | Ovarian | Adenocarcinoma | |
| | CO | | | |
| MLS | U of | | Adenocarcinoma | |
| | Rochester | | | |

The invention provides novel therapeutic formulations, their compositions, methods for their preparation, their physical chemical characteristics and their mode of uptake by cancer cells and tumors and mode of administration as a therapeutic agent. In one aspect, the invention involves the delivery of therapeutic/imaging agents as a component of a nanoparticle that is composed of three lipids and a lipid binding protein. The nanoparticle is spherical in shape with a hydrophobic interior core region that accommodates the water insoluble therapeutic agents (see FIG. 1).

In another preferred embodiment, the chemotherapeutic agent is paclitaxel or doxirubicin. In yet another embodiment, it is valrubicin or doxorubicin. The nanoparticles may be spherical in shape with an average diameter of about 7 nM to about 21 nM.

In a preferred embodiment, the structural components include a phospatidylcholine, a cholesterol, and a cholesteryl ester, as well as a lipid binding protein (an apolipoprotein).

In some embodiments, particles can be prepared that contain conjugates of the apolipoprotein and specific targeting agents (e.g. folate) that will target the drug delivery nanoparticle toward specific cancer cells and tumors, upon being covalently attached to the apolipoprotein/peptide component (see FIG. 2). In another embodiment, nanoparticles can be prepared using small synthetic peptides that may serve as surrogates for Apo A-I.

In another aspect, a pharmaceutical formulation is provided that includes a delivery vehicle combined with a drug in a pharmaceutically acceptable manner. A method for administering a pharmaceutical to an individual is also provided utilizing a formulation composed of a drug and a carrier made up of biocompatible components. In some aspects, the administration is intravenous or parenteral although intramuscular, transmucosal, or transdermal administration is also compatible with the formulation. Because of the numerous opportunities for the surface modification of the delivery vehicle, in some embodiments, the encapsulated pharmaceutical may be formulated for controlled release.

In one embodiment, a method is provided for treating cancerous growths with paclitaxel as the encapsulated pharmaceutical agent, in a pharmaceutically acceptable formulation. In another embodiment, a method is provided for treating a malignant tumor in an individual with a chemotherapeutic agent (such as valrubicin), regarding its physical/chemical properties that will impact its ability to be incorporated into the synthetic high density lipoprotein (sHDL) nanoparticle. In this regard, the solubility of the drug to be incorporated into the carrier particle is crucial as it has to possess sufficient hydrophobicity (water repellent properties) to be appropriately targeted for the core of the carrier complex (FIG. 1). This feature also assures that sufficient amounts of the pharmaceutical agent may be incorporated into the formulation for administration into the individual.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows loading of a hydrophobic drug (paclitaxel) into the core region of the synthetic HDL (s-HDL) nanoparticle;

FIG. 2 shows attachment of targeting signals by covalent linkage to the amino acid side chains of apolipoprotein A-I;

FIG. 3 shows structural comparison of liposomes and synthetic high density lipoprotein (sHDL)/drug complexes;

FIG. 4 shows electron micrograph of the sHDL/PTX nanoparticles;

FIG. 5 shows transformation of the discoidal precursor (nascent) HDL to its stable, spherical configuration via the accumulation of cholesteryl esters. From: Alexander, E. T. et al, Biochemistry, 2005, 44(14):5409-19;

FIG. 6 shows impact of folate attachment to apo A-I on the efficacy of the synthetic HDL particle to deliver paclitaxel to ovarian cancer cells;

FIG. 7 shows uptake of paclitaxel encapsulated in sHDL nanoparticles;

FIG. 8 shows the impact of the sHDL encapsulated valrubicin on ovarian cancer cells (A) and normal spleen cells (B);

FIG. 9 shows retention of sodium cholate and paclitaxel by the sHDL delivery particles;

FIG. 10 shows preparative ultracentrifuge pattern of the sHDL/Taxol®formulation, developed in a discontinuous KBr gradient (d=1.0-1.3 g/ml). The data show no radioactivity outside the isolated sHDL/Taxol® peak indicating no losses due to leakage from the preparation;

FIG. 11 shows gradient gel electrophoresis of the PTX containing delivery particles, with plasma HDL controls (HDL₂ and HDL₃).

FIG. 12 shows re-chromatography of the delivery particles, previously isolated from the same AcA-34 gel column;

FIG. 13 shows comparison of the breast cancer cell (MCF7) killing ability of the PTX encased in the delivery vehicle vs the free drug; and

FIG. 14 shows weight loss incurred by female C57B1/6 mice upon 3 consecutive injections of PTX as Taxol®, Abraxane® and the sHDL/PTX formulation;

FIG. 15 shows a comparison of PTX toxicity toward PC-3 (prostate cancer) cells, formulated by Cholate Dialysis vs. Sonication;

FIG. 16 shows tolerance of female C5TBL6 mice; 6-8 weeks, 18-21 g, when injected with TAXOL® (30 mg/kg paclitaxel); and

FIG. 17 shows tumor suppression studies of TAXOL® and rHDL/PTX.

### DETAILED DESCRIPTION OF PREFERRED EMBODIMENTS

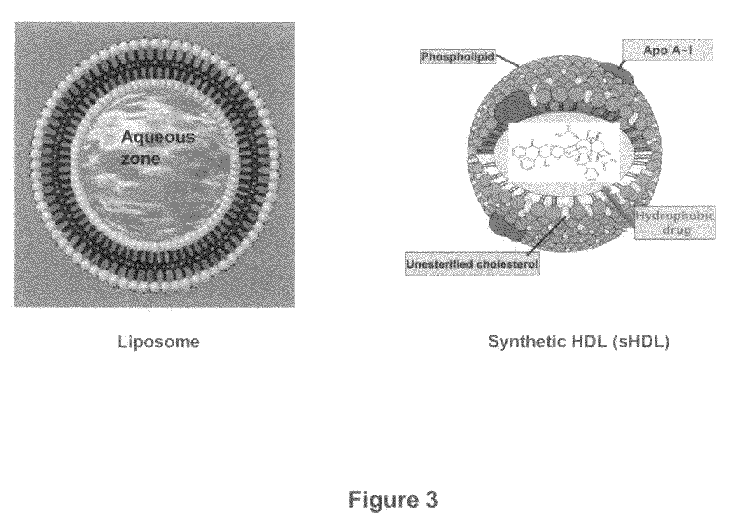

The invention provides compositions and methods for delivery of a pharmaceutical to an individual. Delivery vehicles are provided in a formulation of a pharmaceutical that is encapsulated in a synthetic self-assembled nanoparticle that includes a lipid binding protein and a lipid monolayer. The interior of the particle represents a hydrophobic core region where highly insoluble drug molecules lipid molecules may be incorporated. In contrast to liposomes, that include an aqueous interior core surrounded by phospholipid bilayer, the drug carrier nanoparticle described here is composed of a monolayer and a hydrophobic interior (FIG. 3).

The hydrophobic nature of the interior of the sHDL particle of the invention allows the encapsulation of hydrophobic molecules, in a manner similar to the native core component of HDL (cholesteryl esters). The character of those compounds that are appropriate candidates for encapsulation, including, anti-cancer drugs and imaging agent may be defined by their by their octanol/water partition coefficient X log P (Wang et al. Chem. Inf Comput. Sci. 1997, 37, 615-621). The coefficient for paclitaxel is 3 and for valrubicin is 3.3. We consider all drugs with X log P of above 2 to be excellent candidates for incorporation into sHDL. This characteristic includes over half of the approved pharmaceuticals currently employed for parenteral administration.

In one aspect, the invention provides a synthetic self-assembled “nanoparticle” (also termed “delivery particle” or sHDL/drug complex) that includes a lipid monolayer comprising a phosphatidylcholine (or similar amphipathic lipid), and one or more pharmaceutical agents. In some embodiments, a delivery particle may include one or more types of sphingomyelin or ether phospholipids. In one embodiment, the composition of a pharmaceutical formulation is provided that includes a drug and a pharmaceutically acceptable carrier.

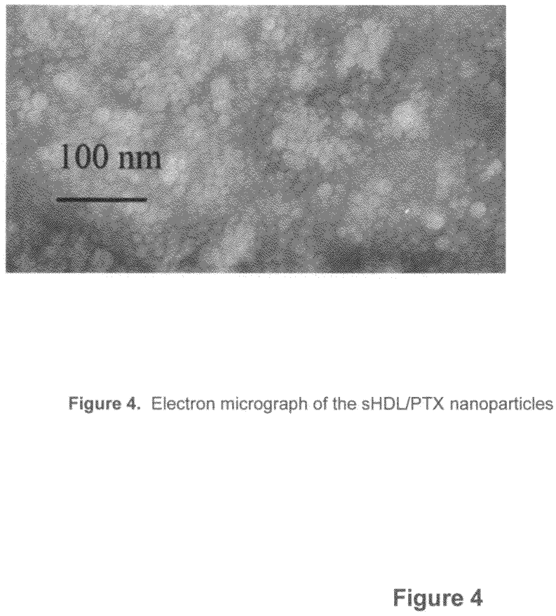

“Self assembly (self assembled or self assembling)” in the case of the generation of rHDL nanoparticles means that the ingredients (such as lipids and proteins) or relatively low molecular weight (such as apo A-I with the molecular weight of 28,000) assembled into a particle of larger molecular weight (such as average molecular weight of about 180,000 or larger) without the application of a physical force, such as sonication, high pressure, membrane intrusion, or centrifugation. The advantages of self assembly from the standpoint of cancer chemotherapy are at least twofold: (1) the pharmaceutical agent incorporated into the self-assembled vs. the sonicated particle favors the former by over 20 fold. (See, Lacko et al., “High Density Lipoprotein Complexes as Delivery Vehicles for Anticancer drugs,” Anticancer Research 22: 2045-9 (2002); and Corbin et al. “Enhanced Cancer-Targeted Delivery Using Engineered High-Density Lipoprotein Based Nanocarriers,” J. Biomed Nanotech. 3:367-76 (2007), published in August 2007.). This increase of incorporation is a substantial advantage because of the substantially increased effective cytotoxicity of the self assembled particles toward cancer cells and tumors, and (2) the increased uniformity of the self assembled particles vs. those generated by physical force (such as sonication). (See, FIG. 4 showing the uniformity of the self assembled particles). This uniformity is also advantageous from the standpoint of more efficient delivery of the drug, encapsulated in the nanoparticles, to tumors.

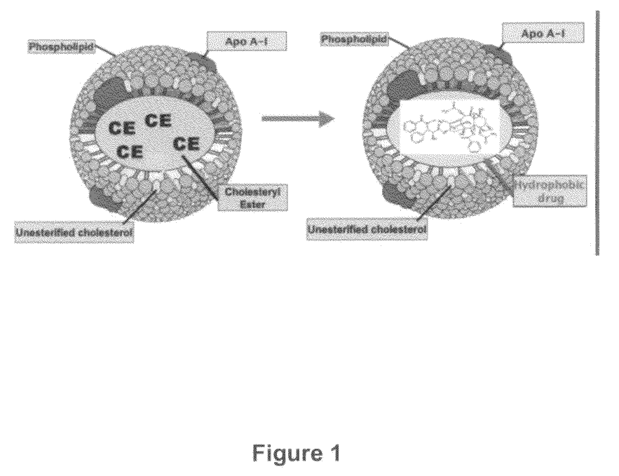

The interior of a particle includes a hydrophobic core region where the transported materials reside in a manner similar to the native cholesteryl esters in HDL (FIG. 1). Particles of the invention do not include a hydrophilic or aqueous core. The particles are generally of spherical shape with phospholipids and α-helices or β-sheets of the lipid binding proteins that are associated with hydrophobic surfaces of the monolayer around the interior core region (FIG. 1). The diameter of a typical spherical sHDL/drug delivery nanoparticle is about 7 nm to 16 nm; minimally 7 nm and maximally 21 nm. Based on the incorporation of higher amounts of a drug, this diameter may expand to 40-50 nm.

The sHDL/drug nanoparticles may be utilized to deliver a drug or anti-cancer agent to an individual. Pharmaceutical agents, incorporated into the sHDL delivery particles, as referred to here, generally include at least one hydrophobic region capable of being incorporated into the hydrophobic core region of the carrier particle (FIG. 1). In some embodiments, originally hydrophilic drugs are modified to increase their hydrophobicity (Versluis et al, J Pharmacol Exp Ther. 1999 289:1-7 and Lee et al, Chemotherapy 2005; 51:311-318) and thus allow their efficient incorporation into the core of the delivery particles. Because of the natural components of the delivery particle, the complex formed by the encapsulation of the pharmaceutical agent is substantially nonimmunogenic when administered to an individual.

### Lipid Monolayer

The delivery particles of the invention include a lipid monolayer with the polar head groups of phospholipids facing away from the interior of the particle, and a hydrophobic core region where the pharmaceuticals are encapsulated (FIG. 1).

Any monolayer-forming lipid may be used that along with a lipid binding protein forms the scaffolding for the spherical particle to accommodate the drug to be transported in the interior of the particle. The term “monolayer-forming lipid” refers to a compound that is capable of forming a lipid monolayer serving as an outer shell of the basic lipoprotein structure (FIG. 1A). In some embodiments, the lipid monolayer is made up of phosphatidylcholine. Examples of this include but are not limited to dimyristoyl PC (DMPC), dioleoyl-PC (DOPC), dipalmitoylphosphatidylcholine (DPPC), or other phospholipids such as, egg yolk phosphatidylcholine (egg PC), soy bean phosphatidylcholine etc. In another embodiment sphingomyelin, cationic phospholipids or glycolipids may be used to form the monolayer to produce delivery particles with additional properties. Particles able to perform controlled release of the encapsulated pharmaceutical could be prepared using these latter ingredients.

### Lipid Binding Proteins

The term lipid binding protein, as used here, refers to synthetic or naturally occurring peptides or proteins that are able to sustain a stable complex with lipid surfaces and thus able to function to stabilize the lipid monolayer of the nanoparticle of the invention. The sHDL/drug nanoparticles may include one or more types of lipid binding proteins or apolipoproteins that are natural components of plasma lipoproteins (Ajees et al; Proc Natl Acad Sci USA. 2006 103:2126-31). In some embodiments, nanoparticles can be prepared using small synthetic peptides that may serve as surrogates for apo A-I (Navab et al Arterioscler Thromb Vasc Biol. 2005 25:1325-31) and thus yield formulations with additional properties once incorporated into the sHDL/drug complex.

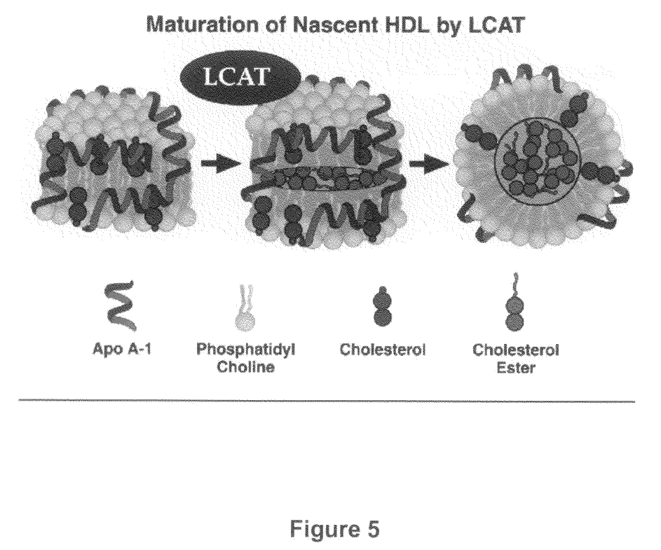

Apolipoproteins generally include a high content of amphipathic α-helix motif that facilitates their ability to bind to hydrophobic surfaces, including lipids. An important characteristic of apolipoproteins is to support the structure of monolayers, vesicles or bilayers, composed primarily of phospholipids and to transform them into disc-shaped complexes (Saito et al Prog Lipid Res. 2004, 43:350-80). Subsequently, under physiological conditions, the discoidal complexes undergo a transition to a spherical structure (Alexander, E. T. et al, Biochemistry, 2005, 44:5409-19), facilitated by the enzyme lecithin cholesterol acyltransferase (LCAT) to produce HDL (FIG. 5).

The particles of the invention are also spherical and contain ingredients similar to those of HDL. However, these spherical shaped, drug containing structures are generated by a self-assembly process that does not involve any physical force such as sonication or membrane extrusion.

The drug carrying nanoparticles contain one or more pharmaceutical agents. The term “pharmaceutical agent” or “drug” as used herein refers to any compound or composition having preventive, therapeutic or diagnostic activity, primarily but not exclusively in the treatment of cancer patients.

The invention describes lipid binding polypeptides, which are used to prepare the delivery particles described above. These lipid binding proteins may include one or more attached “functional moieties,” including, one or more targeting signals, with a specific desired biological activity, that may be capable of augmenting the efficacy of a pharmaceutical agent incorporated into the delivery particle. In one embodiment, the moiety may enhance the targeting of the encapsulated drug via interaction of the carrier particle with a cell surface receptor specifically expressed by a cancer cell or tumor.

In some embodiments, the lipid binding peptide or protein can be a synthetic analog or surrogate (Navab et al Nat Clin Pract Cardiovasc Med. 2006; 3:540-7) for the naturally occurring apolipoprotein (apo A-I) that is used in the preparation of the carrier particles.

### Modified Lipid Binding Polypeptides

In some embodiments of the invention, a lipid binding protein (apo A-I) is used following chemical modification so that when the modified apo A-I is used as a component of the drug carrying delivery particle, it will have increased targeting ability.

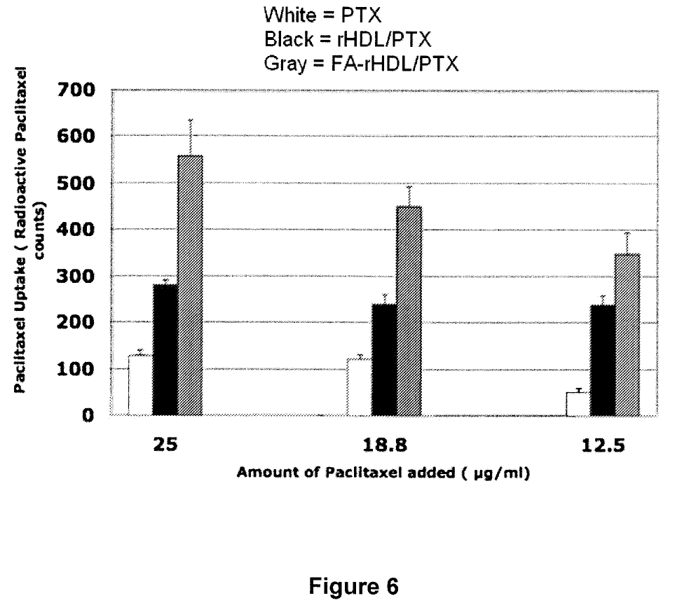

In one example, the apo A-I protein is modified by the attachment of folic acid residues that results in the doubling of the drug uptake by ovarian cancer cells compared to the non-modified formulation (FIG. 6).

### Delivery System for Delivery of a Bioactive Agent to an Individual

This approach provides a system comprised of delivery particles as a pharmaceutically acceptable formulation for delivering pharmaceutical. particularly anti-cancer drugs to an individual. In some embodiments, the delivery system comprises an effective therapeutic approach to kill cancer cells or to destroy malignant tumors. In other embodiments, the delivery system comprises an effective imaging approach to identify the presence or absence of malignant tumors.

The term “individual” as used herein refers to any cell, animal tissue, or a vertebrate animal. In some embodiments, the individual is a vertebrate, such as a human, a nonhuman primate, or an experimental animal, such as a mouse or rat. In some embodiments, delivery particles are formulated as a carrier, suitable for administration to an individual. The term “carrier” as used herein, refers to a biocompatible nanoparticle that facilitates administration of a pharmaceutical agent to an individual.

The term “effective amount” as used herein refers to the amount of a pharmaceutical sufficient to bring about the desired results in an experimental setting. A “therapeutically effective amount” or “therapeutic dose” refers to an amount of a pharmaceutical that is sufficient to produce beneficial clinical results, such as reduction in tumor size or remission for cancer patients.

The term “nanoparticle” as used herein refers to a particle with a diameter of less than about 1000 nm.

### Methods of Use

The invention describes approaches for administering a pharmaceutical agent to an individual. The methods of the invention include preparing and administering a delivery particle as described above that includes a lipid binding polypeptide, a lipid monolayer, and a drug that is enclosed in the interior core compartment of the synthetic lipoprotein particle (FIG. 1).

Optionally, therapeutically effective amounts of the delivery particles are administered, in a pharmaceutically acceptable formulation. Generally, the particles are spherical with a diameter of about 7 to about 21 nm, as measured by transmission electron microscopy (FIG. 4). Typically, the pharmaceutical agent has at least one hydrophobic region that facilitates its integration into a hydrophobic core region of the carrier particle.

The route of administration of the drug, enclosed in the carrier particle, may vary according to the nature of the pharmaceutical agent to be administered or the condition to be treated. For mammals, the administration is generally parenteral. Routes of administration include, but are not limited to intravenous, intramuscular, subcutaneous, transmucosal, and transdermal. Delivery particles may also be formulated for controlled release. The term “controlled release” as used herein refers to release of a drug from the carrier particle so that the blood or tissue levels of the pharmaceutical is maintained within the desired therapeutic range for an extended period (hours or days).

In one aspect, the invention provides a method for treating cancerous growth or a tumor in an individual. The method includes administering a therapeutically effective amount of a chemotherapeutic agent enclosed in the delivery particles as described above (FIG. 1) in a pharmaceutically acceptable formulation. In one embodiment, the chemotherapeutic agent is paclitaxel. In addition, the lipid binding protein component of the delivery particles may include a targeting moiety to target the particles to tumor cells (FIG. 6). In one embodiment, folic acid is attached to the lipid binding protein (apo A-I or its fragment).

### Targeting

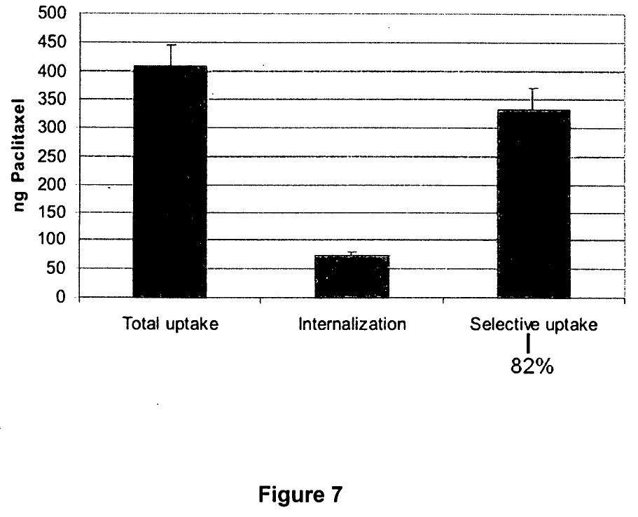

The delivery particle of the invention may include a targeting function as the lipid binding protein component (apo A-I) is the natural ligand for the HDL receptors (scavenger receptor type B-1-SR-B1 and CD36 and LIMPII Analogous-1-CLA-1). This receptor system allows the selective uptake of the natural core component, cholesteryl ester from HDL. During our studies, we have shown that the drug paclitaxel is also taken up by cancer cells via this receptor mediated mechanism, when encapsulated in the delivery particles presented by this invention (FIG. 7).

In some embodiments, involving the treatment of malignant tissues, targeting is major advantage because most cancerous growths have been shown to have enhanced receptor expression and thus would favor the uptake of the drug that is encased in the delivery particles vs normal tissues and thus would reduce the danger of side effects.

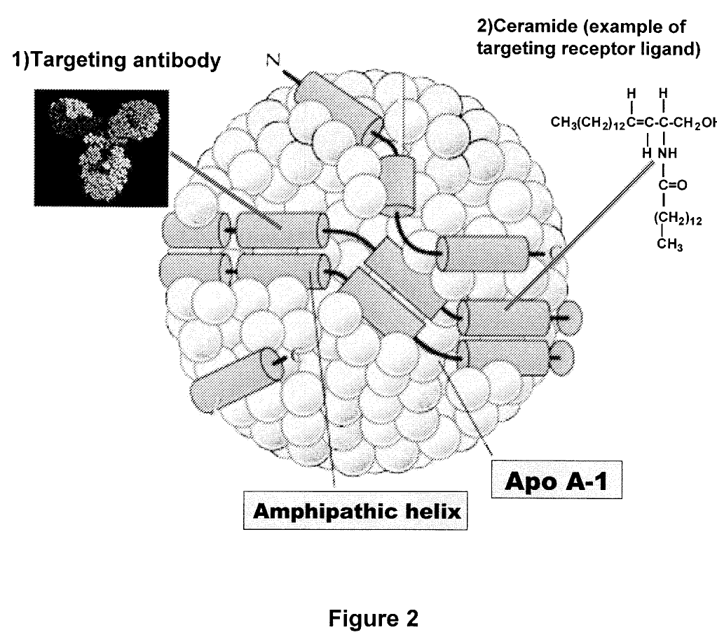

In other embodiments, additional receptor binding components may be attached to a lipid binding protein component (apo A-I) to enhance the targeting potential of the delivery vehicle. In one embodiment, folate was used to demonstrate the potential of this approach (FIG. 6) because folate receptors are upregulated in most ovarian tumors. Because nearly all cancer cells feature substantially higher expression of one or more specific surface antigens, ultimately individual therapy of patients will be possible following a proteomic screen of the tumor (Calvo et al, Biosci Rep. 2005, 25:107-25). In another embodiment, the lipid binding protein moiety of the delivery particle may be modified to produce specifically targeted therapeutic strategies (FIG. 2).

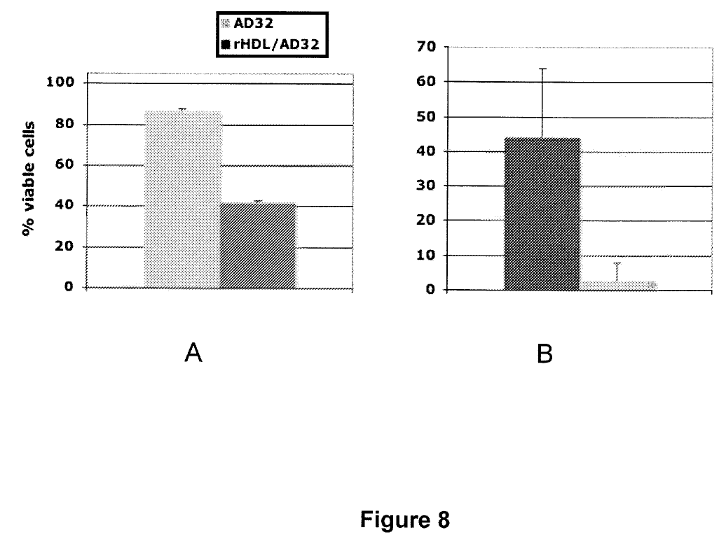

In yet another embodiment, the drug valrubicin, encapsulated in the delivery particles, may be used to kill cancer cells or tumors more effectively than the free drug (FIG. 8) while the impact of the drug on normal cells is substantially reduced, when encapsulated.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation and Characterization of sHDL/Paclitaxel Particles

Preparation of Recombinant ApoA-I. Recombinant Apo-A-I is prepared as described in Ryan et al. (2003) Protein Expression and Purification 27:98-103, and was used to prepare sHDL/paclitaxel complexes.

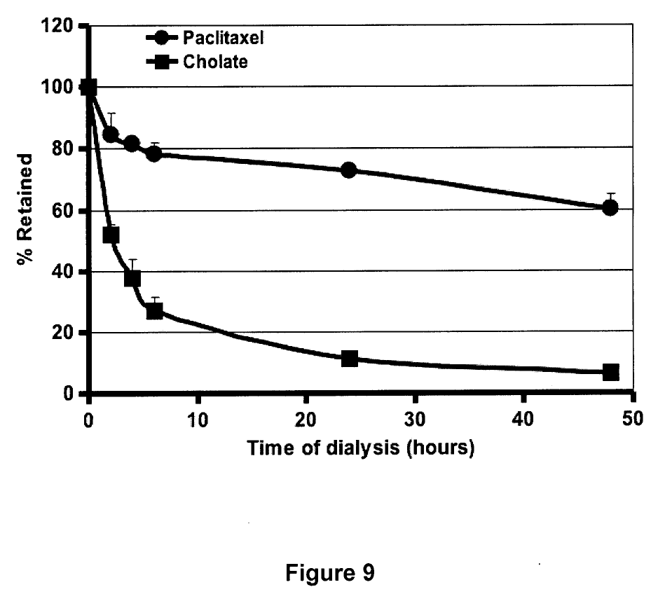

The particles are prepared by a process involving cholate dialysis as described below to produce a spherical structure with the pharmaceutical enclosed in the interior hydrophobic core region. The lipid mixture (egg yolk phosphatidylcholine, cholesterol and cholesteryl oleate in the ratio of 3.8:1:88.5) and 2 mg paclitaxel is dried under N₂ to a thin film and dispersed in dimethylsulfoxide and subsequently in 1.4 ml of 10 mM Tris, 0.1 M KCl, 1 mM EDTA, pH 8.0). Sodium cholate, 140 μl (100 mg/ml stock in [0.15 M NaCl 0.003 M KCl, 0.15 M KH₂PO₄, pH 7.4, designated as PBS]) is added to produce mixtures with a final PC to cholate molar ratio of ˜1:1.6. Apo A-I (12.7 mg/ml) in 0.4 ml of PBS is added to this mixture and the final volume is adjusted to 2 ml with PBS. The lipid/protein/cholate mixture is then incubated for 12 hrs at 4° C., followed by dialysis (2 liter of PBS, for two days) with three buffer changes. Using ³H-cholate as a tracer, <2% of the cholate remained in the sHDL/drug preparations while over 60% of the paclitaxel remained associated with the sHDL delivery particles (FIG. 9).

### Storage and Stability

Particles of the invention can be stored at 4° C. and remain stable for at least 60 days.

### Example 2

### Characterization of Particles

The delivery particles, containing paclitaxel (sHDL/paclitaxel) had a very consistent composition that varied less than 5% from batch to batch. The typical composition of the delivery particle containing paclitaxel (sHDL/paclitaxel) is shown in Table 4.

TABLE 4
Composition of the delivery particles, containing
paclitaxel (sHDL/paclitaxel).

| | Protein | PC | FC | CE | TC | Ptx |
|---|---|---|---|---|---|---|
| | mg/ml | | | | | |
| Amount | 1.65 | 1.35 | 0.09 | 0.05 | 0.14 | 0.45 |
| incorporated | | | | | | |
| Distribution | 46.0% | 37.7% | 2.5% | 1.4% | 3.9% | 12.4% |
| % | | | | | | |

PC = phosphatidylcholine (egg yolk), FC = free (unesterified cholesterol), CE = cholesteryl ester (cholesteryl oleate). Ptx = paclitaxel.

Comparison of the PTX and Cyclosporine Carrying Capacity of the Present Invention with Another sHDL Formulation

TABLE 5
Comparison of the drug carrying capacity of sonicated
(Sparks/Ptx and Sparks/CycA) vs non-sonicated (sHDL/Ptx
and Sparks/CycA) delivery particles.

| Formulation | Drug | Protein | PL | TC |
|---|---|---|---|---|
| sHDL/Ptx | .37 ± .02 | 2.41 ± .06 | 4.53 ± .07 | .16 ± .01 |
| Sparks/Ptx* | .034 ± .001 | .42 ± .02 | .32 +/− .03 | N/A |
| sHDL/Cyc A | .41 ± .004 | 2.7 ± .08 | 4.77 ± .28 | .15 ± .01 |
| Sparks/Cyc A* | .058 ± .002 | .70 ± .05 | .74 +/− .04 | N/A |

*“Sparks” is a particle formed by sonication as reported in U.S. Pat. No. 6,514,523.

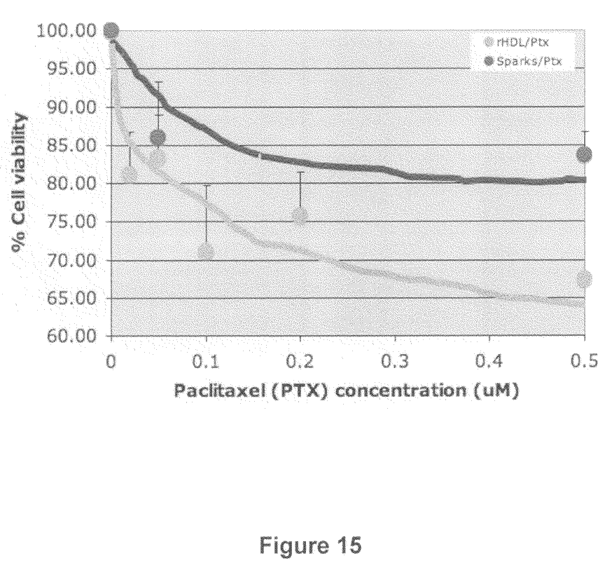

Delivery particles containing paclitaxel were prepared as described above. In addition another formulation was used employing the method disclosed in U.S. Pat. No. 6,514,523. The data shown in Table 5. clearly favor the preparation disclosed in this document (formulated without the use of physical force, sonication) as 8-10 times more drug was incorporated into these particles as compared to the incorporation efficiency of the sonicated particles. FIG. 15 shows a comparison of PTX toxicity toward PC-3 (prostate cancer) cells, formulated by cholate dialysis vs. sonication.

### Characterization

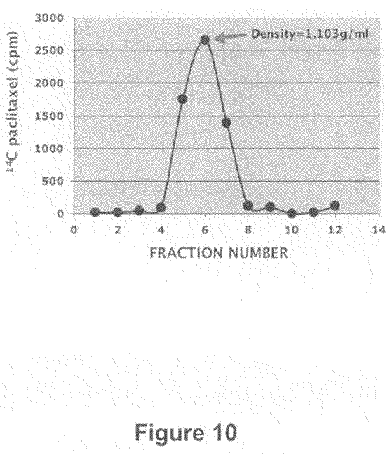

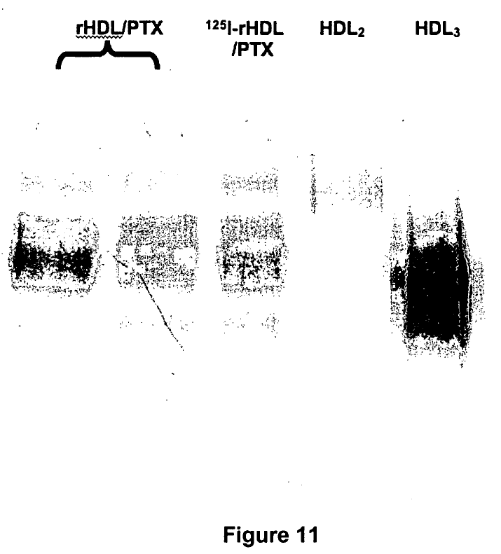

Characterization studies revealed that the paclitaxel containing delivery particles float as a discrete particle population when subjected to density gradient ultracentrifugation (FIG. 10), with a density characteristic of high density lipoproteins. Further, gradient gel electrophoresis under non-denaturing conditions revealed that the major component of the drug carrying sHDL particles migrated between the HDL controls (HDL₂ and HDL₃, [FIG. 11]). Thus no disintegration of the delivery particle or leakage of the drug was observed under any of these conditions tested, testifying to its robustness of the sHDL nanoparticles.

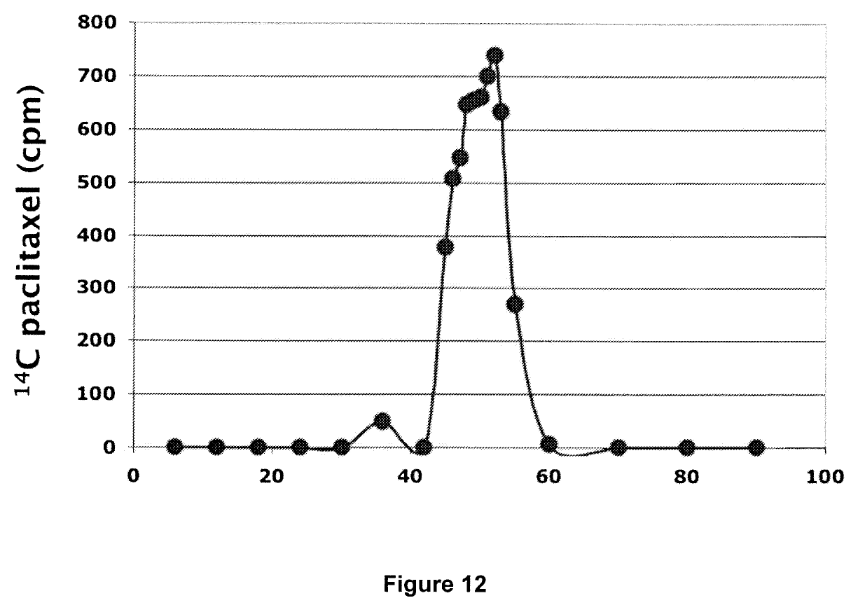

Molecular weight of the delivery particles was estimated to be 180-200,000 using gel exclusion chromatography (FIG. 12). The data from these experiments, along with the other findings, shown above (FIGS. 4, 7, 10, 11 and Table 1) clearly show that the delivery particles described here represent discrete nanoparticle entities, composed of bio-compatible ingredients that resemble high density lipoproteins.

In addition to providing information on the molecular weight of the delivery particles, the data show no losses due to leakage, once again attesting to the stability of the sHDL/drug formulation.

### Cytotoxicity

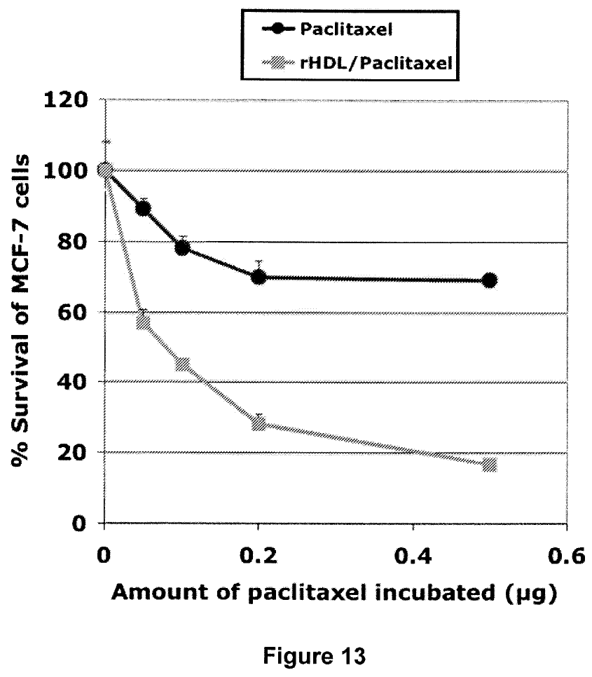

As shown above (FIG. 8), in one embodiment, the valrubicin containing delivery particles were highly effective in killing cancer cells while protecting against toxicity to normal cells. In another embodiment, the paclitaxel (PTX) containing delivery particles were also found to be highly effective in killing breast cancer (FIG. 13.) and other cancer cells (Table 6).

TABLE 6
Comparisons of IC50 values for cancer cell lines
between the PTX encapsulated in the delivery particle
(sHDL) vs those for the free drug

| | IC₅₀ for rHDL/Ptx | IC₅₀ for Ptx |
|---|---|---|
| Cell lines used | (μM) | (μM) |
| OV1063 | 1.2 | 14.2 |
| OVCAR 3 | 14.1 | 70.3 |
| DU145 | 1.8 | 14.2 |
| MCF7 | 0.6 | 14.2 |

In Vivo Assessment of Toxicity (maximum tolerated dose)

### Animals

Female C57BL6 mice (6-8 weeks, 18-21 g) were housed no more than four per cage in accordance with Institutional Animal Care and Use Committee guidelines.

### Toxicity Study

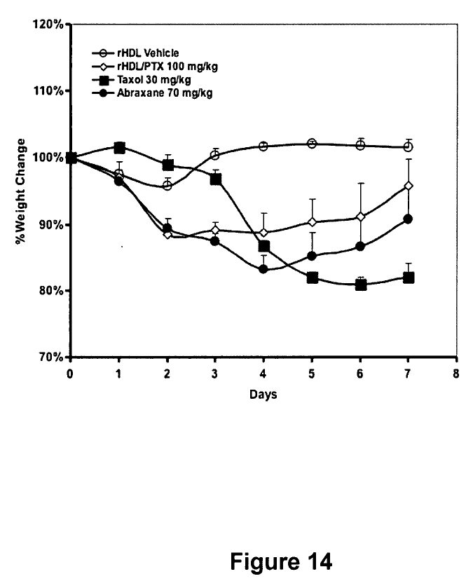

Groups of six mice each received injections of 1.5 ml of phosphate buffered saline (PBS) via the intraperitoneal route, containing respective doses of 30 mg/kg and 40 mg/kg of Taxol®, 40 mg/kg and 70 mg/kg of Abraxane® and 85 mg/kg and 100 mg/kg of sHDL/Ptx. The data presented in FIG. 14 show that the sHDL/PTX formulation proved to be markedly superior to both Abraxane® and Taxol®. Accordingly, the estimated MTD (based on the 15% weight loss criteria) was 30 mg/kg for Taxol® and 70 mg/kg for Abraxane®, the sHDL/PTX formulation did not reach the 15% weight loss even at the 100 mg/kg dosage of PTX.

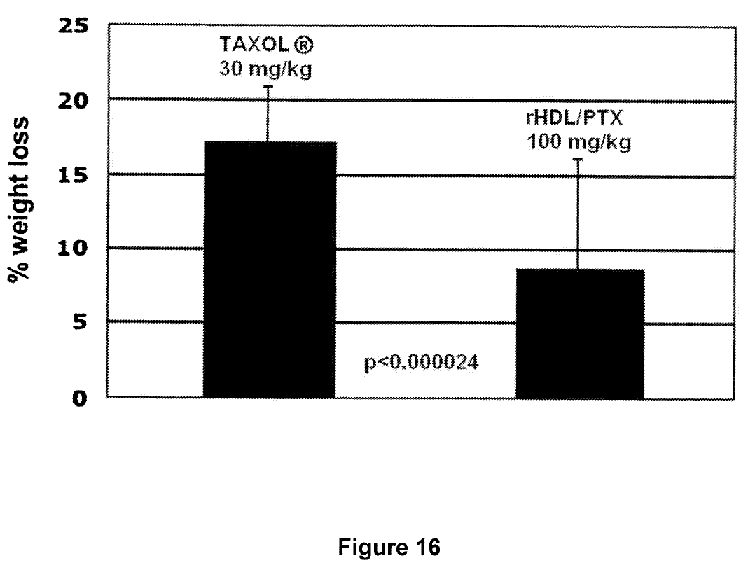

FIG. 16 shows that TAXOL® injected at 30 mg/kg caused more body weight loss than sHDL/PTX injected at 100 mg/kg in mice (female C57BL6, 6-8 week, 18-21 g), indicating that animal could tolerate more of PTX when it is encapsulated in the sHDL of the present invention.

### Tumor Suppression Study

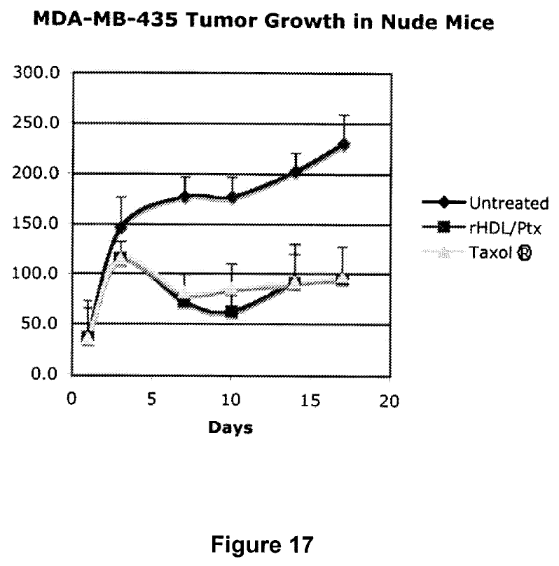

FIG. 17 shows the result of tumor suppression studies. Each group contained 5 mice (Mouse/nu/nu strain, from Harlan, 4-5 weeks old and about 18-22 grams). Each animal was implanted subcutaneously (“SC”) with cells harvested from tissue culture of MDA-MB-435 breast cancer cells. When tumor grew to approximately 125 mm³ (100-150 mm³), animals were pair-matched by tumor size into treatment and control groups. Here the suppression of tumor growth was about the same for TAXOL® (30 mg/kg paclitaxel) and sHDL/PTX (80 mg/kg paclitaxel). TAXOL®, however, is formulated with a powerful detergent Cremophor that in itself is cytotoxic and is also the source of numerous side effects during chemotherapy. The Cremophor content of TAXOL® is about 80× that of paclitaxel per ml. In light of the fact that animals can tolerate more of rHDL/PTX as compared to TAXOL®, it is safe to say that rHDL/PTX would have more effective antitumor efficacy than TAXOL®.

### Example 3

### HDL Preparation

Two sources of HDL are available in bulk quantities as HDL apolipoproteins, including apoA-1. Bulk quantities of HDL may be prepared from salvage plasma or from blood product supernatants (such as a by-product of the cryoprecipitation scheme at blood banks), according to a procedure developed in the inventors laboratory. The procedure appears in Lacko, A. G. and Chen, T. F., J. Chromatog, 130: 446-450 (1977) and is specifically incorporated herein by reference for this purpose. This method allows the isolation of HDL from plasma in excellent yield, and may be scaled to industrial production levels for production of apoA-I or other apolipoproteins found in HDL. The HDL preparations can be subjected to Heparin-Sepharose chromatography to remove the apoE-containing fraction. The removal of apoE should enhance the specific uptake of the drug-HDL complexes by tumor cells vs non-malignant cells. The second source is the procurement of Cohn fraction IV, a by-product of albumin preparation and other serum proteins.

### Example 4 Cell Killing Potential of HDL/Drug Preparations

The cytotoxic effect of each HDL/drug preparation on breast cancer cells to be assessed by measuring the conversion of the tetrazolium dye MTT to formazan in a microtiter plate format.

The assessment of PTX and toxicity is performed as follows: Cells are grown to 80-90% confluency and detached from the flask by digesting with 0.25% Trypsin (no EDTA). The cells will be subsequently washed from the flask with complete media, and then centrifuged for 5 minutes at 1000 g to pellet the cells. The cells are then resuspended in complete media. An aliquot is obtained and diluted 1:1 with Trypan Blue. The cells are counted and their viability determined according to the following procedure. The cells are seeded in 96 well microtiter plates (at 3000 cells per well) and allowed to attach for 24 hours. Taxol is diluted to yield a stock solution of 5 μg per 1 ml of PBS and added in 40 μl aliquots to each of the appropriate wells (total volume of 120 μl/well). Controls include media+PTX. Following a 72-96 hour exposure to PTX and sHDL-PTX complexes, 12.5 μl of the 10 mg/ml of MTT stock solution is added to each well. After a 2 hour incubation at 37° C. and 5.0% CO₂, 100 μl of lysing buffer is added to each well. The plates were incubated at 37° C. and 5.0% CO₂ overnight, removed from the incubator and then allowed to cool for 15 minutes at room temperature before measuring the absorbance values at 595 nm.

### Example 5

### Screening of Cancer Cell Cultures for HDL/Drug Complex Incorporation

Cultured cells are incubated with the sHDL/PTX complex, labeled with ¹⁴C-PTX. Subsequent to the incubation period, cells are trypsinized and the radioactivity of the lysate is determined to measure the extent of incorporation of the PTX into the cells (FIG. 7).

Although the invention described in some detail in the foregoing, by way of illustration and examples for purposes of clarity of understanding, it will be apparent to those skilled in the art that certain changes and modifications may be employed without departing from the spirit and the scope of the invention. Therefore, the description should not be construed as limiting the scope of the invention, delineated by the appended claims.

### REFERENCES CITED

The following references, to the extent that they provide exemplary procedural or other details supplementary to those set forth herein, are specifically incorporated herein by reference.

### U.S. Patent Documents

U.S. Pat. No. 6,998,388 to Cockerill et al.
U.S. Pat. No. 6,117,454 to Kreuter, et al.
U.S. Pat. No. 7,053,049 to Luescher et al.
U.S. Patent Publication 2004/0204354 by Nelson et al.
U.S. Patent Publication 2004/0229794 by Ryan and Oda
U.S. Patent Publication 2003/0008014 by Shelness
U.S. Pat. No. 6,514,523 to Sparks
U.S. Pat. No. 5,652,339 to Lerch, et al.
U.S. Pat. No. 5,128,318 to Levine, et al.
U.S. Pat. No. 5,089,602 to Isliker, et al.

### Foreign Patent Documents

PCT Patent Publication No. WO/1998/046275 by Counsell and Pohland describes a blood-pool carrier for lipophilic imaging agents.
PCT Patent Publication No. WO/2006/073419 by Glickson et al. describes lipoprotein nanoplatforms.
PCT Patent Publication No. WO/1998/013385 by Halbert et al. describes non-naturally occurring lipoprotein particles.
PCT Patent Publication No. WO/1986/07539 by Masquelier et al. describes a method for the production of a macromolecular carrier loaded with a biologically active substance.

### Non Patent Literature Documents

Ajees et al; Proc Natl Acad Sci USA. 103:2126-31 (2006)
Alexander, E. T. et al, Biochemistry, 44:5409-19 (2005)
Bijsterbosch M K, Ziere G J, Van Berkel T J. Lactosylated low density lipoprotein: a potential carrier for the site-specific delivery of drugs to Kupffer cells. Mol Pharmacol September; 36(3):484-9 (1989)
BIJSTERBOSCH M K, et al., Synthesis of the dioleoyl derivative of iododeoxyuridine and its incorporation into reconstituted high density lipoprotein particles. Biochemistry. November 29; 33(47):14073-80 (1994)
BLANCHE P J, Nichols A V, Forte T M, Gong E L. Characterization of Complexes of Egg Yolk Phosphatidylcholine and Apolipoprotein A-II Prepared in the Absence and Presence of Sodium Cholate. Biochim Biophys Acta. February 4; 958(2):143-52 (1988)
Brundert M, Heeren J, Bahar-Bayansar M, Ewert A, Moore K J, Rinninger F. Selective uptake of HDL cholesteryl esters and cholesterol efflux from mouse peritoneal macrophages independent of SR-BI. J Lipid Res. 47:2408-21 (2006).
Chung N S, Wasan K M. Potential role of the low-density lipoprotein receptor family as mediators of cellular drug uptake. Adv Drug Deliv Rev. 56:1315-34 (2004).
Counsell R E, Pohland R C. Lipoproteins as potential site-specific delivery systems for diagnostic and therapeutic agents. J Med. Chem. 25:1115-20 (1982).
Calvo K R, Liotta L A, Petricoin E F, Clinical proteomics: from biomarker discovery and cell signaling profiles to individualized personal therapy. Biosci Rep. 25:107-25 (2005).
Edwards I. J., Berquin I. M., Sun H., O'flaherty J. T., Daniel L. W., Thomas M. J., Rudel L. L., Wykle R. L., Chen Y. Q. Differential effects of delivery of omega-3 fatty acids to human cancer cells by low-density lipoproteins versus albumin. Clin Cancer Res, 10:8275 (2004).
Hevonoja T, Pentikainen M O, Hyvonen M T, Kovanen P T, Ala-Korpela M. Structure of low density lipoprotein (LDL) particles: basis for understanding molecular changes in modified LDL. Biochim Biophys Acta. 1488:189-210 (2000).
Ibrahim N K, Samuels B, Page R, Doval D, Patel K M, Rao S C, Nair M K, Bhar P, Desai N, Hortobagyi G N. Multicenter phase II trial of ABI-007, an albumin-bound paclitaxel, in women with metastatic breast cancer. J Clin Oncol. 2005 September 1; 23(25):6019-26.
Jonas, A., Wald, J. H., Toohill, K. L., Krul, E. S., Kézdy, K. E. The conformation of apolipoprotein A-I in discoidal and spherical recombinant high density lipoprotein particles. 13C NMR studies of lysine ionization behavior. J Biol Chem, 265: 22123 (1990).
Kreuter, J., Shamenkov, D., Petrov, V., Ramge, P., Cychutek, K., Koch-Brandt, C., Alyautdin, R. Apolipoprotein-mediated transport of nanoparticle-bound drugs across the blood-brain barrier. J. Drug Targeting, 10: 317-325 (2002).
Lacko, A. G.; Nair, M.; Paranjape, S.; Johnson, S.; McConathy, W. J. High density lipoprotein complexes as delivery vehicles for anticancer drugs. Anticancer Res. 22, 2045 (2002).
Lacko, A. G.; Nair, M.; McConathy, W. J. in. Nanotechnology for Cancer Therapeutics. CRC Press M. Amiji Ed. Chapter 37 (in press).
Lacko, A. G. and Chen, T. F., J. Chromatog, 130: 446-450 (1977)
Lerch, P. G., Fortsch, V., Hodler, G., Bolli, R. Production and characterization of a reconstituted high density lipoprotein for therapeutic applications. Vox Sang, 71: 155, (1996).
Levine D. M., Simon S. R., Gordon B. R., Parker T. S., Saal S. D., Rubin, A. L.; U.S. Pat. No. 5,128,318, 1992.
Lundberg B. Preparation of drug-low density lipoprotein complexes for delivery of antitumoral drugs via the low density lipoprotein pathway. Cancer Res. 47:4105-8. (1987).
Navab M, Anantharamaiah G M, Reddy S T, Van Lenten B J, Wagner A C, Hama S, Hough G, Bachini E, Garber D W, Mishra V K, Palgunachari M N, Fogelman A M. An oral apoJ peptide renders HDL antiinflammatory in mice and monkeys and dramatically reduces atherosclerosis in apolipoprotein E-null mice. Arterioscler Thromb Vasc Biol. 25:1325-31 (2005)
Navab et al Nat Clin Pract Cardiovasc Med.; 3:540-7 (2006)
O'Reilly T, McSheehy P M J, Wenger F, Hattenberger M, Muller M, Vaxelaire J, Altmann K H, and Wartmann M. Patupilone (Epothilone B, EPO906) Inhibits Growth and Metastasis of Experimental Prostate Tumors In Vivo The Prostate 65:231-240 (2005)
Petrak K. Nanotechnology and site-targeted drug delivery. J Biomater Sci Polym Ed.; 17:1209-19 (2006).
Prokai, L., Prokai-Tatrai, K. Prodrugs, Chapter 12 in Pain, Irritation and Muscle Damage with Injectable Products, P. Gupta and G. Brazeau, Eds., Interpharm Press, Denver, Colo., p. 267-306 (1999).
Pussinen P J, Karten B, Wintersperger A, Reicher H, McLean M, Malle E, Sattler W. The human breast carcinoma cell line HBL-100 acquires exogenous cholesterol from high-density lipoprotein via CLA-1 (CD-36 and LIMPII analogous 1)-mediated selective cholesteryl ester uptake. Biochem J. 349:559-66 (2000).
Rhainds D, Brodeur M, Lapointe J, Charpentier D, Falstrault L, Brissette L. The role of human and mouse hepatic scavenger receptor class B type I (SR-BI) in the selective uptake of low-density lipoprotein-cholesteryl esters. Biochemistry. 42:7527-38 (2003).
Ryan et al. Protein Expression and Purification 27:98-103 (2003)
Schouten, D., van der Kooij, M., Muller, J., Pieters, M. N., Bijsterbosch, M. K., van Berkel, T. J. Development of lipoprotein-like lipid particles for drug targeting: neo-high density lipoproteins. Mol Pharmacol, 44:486 (1993).
Saito et al Prog Lipid Res., 43:350-80 (2004)
Segrest J P, Jones M K, Klon A E, Sheldahl C J, Hellinger M, De Loof H, Harvey S C., A detailed molecular belt model for apolipoprotein A-I in discoidal high density lipoprotein J Biol Chem. November 5; 274(45):31755-8 (1999).
Shaw J M, Shaw K V, Yanovich S, Iwanik M, Futch W S, Rosowsky A, Schook L B. Delivery of lipophilic drugs using lipoproteins. Ann N Y Acad. Sci. 507:252-71 (1987).
Sparks, D. L., Phillips, M. C., Lund-Katz, S. The conformation of apolipoprotein A-I in discoidal and spherical recombinant high density lipoprotein particles. 13C NMR studies of lysine ionization behavior. J Biol Chem, 267:25830 (1992).
Versluis et al J Phammacol Exp Ther. 1999 289:1-7 and Lee et al, Chemotherapy 2005; 51:311-318
Wang et al. Chem. Inf Comput. Sci., 37, 615-621 (1997)
Wang M, Briggs M R. HDL: the metabolism, function, and therapeutic importance. Chem Rev. 104:119-37 (2004).
Zheng G, Chen J, Li H, Glickson J D. Rerouting lipoprotein nanoparticles to selected alternate receptors for the targeted delivery of cancer diagnostic and therapeutic agents. Proc Natl Acad Sci USA. 102:17757-62 (2005).
Zorich N L, Kezdy K E, Jonas A. Properties of Discoidal Complexes of Human Apolipoprotein A-I with Phosphatidylcholines Containing Various Fatty Acid Chains. Biochim Biophys Acta June 2; 919(2):181-9. (1987)

What is claimed:

1. A nanoparticle delivery vehicle, comprising:
a self-assembling reconstituted high density lipoprotein complex comprising a combination of:
a) a lipid component;
b) a cholesterol component; and
c) a lipid binding protein component;
wherein the self-assembling reconstituted high density lipoprotein complex has a diameter in the range of from about 7 nm to 100 nm.

2. The nanoparticle delivery vehicle of claim 1, wherein the lipid component comprises a phosphatidylcholine.

3. The nanoparticle delivery vehicle of claim 1, wherein the lipid component comprises a mixture of a phosphatidylcholine, cholesterol, and cholesteryl oleate.

4. The nanoparticle delivery vehicle of claim 1, wherein the lipid component comprises one or more molecules selected from the group consisting of dimyristoyl PC (DMPC), dioleoyl-PC (DOPC), dipalmitoylphosphatidylcholine (DPPC), egg yolk phosphatidylcholine (egg PC), soy bean phosphatidylcholine, sphingomyelin, cationic phospholipids, glycolipids, and any combination thereof.

5. The nanoparticle delivery vehicle of claim 1, wherein the nanoparticle delivery vehicle is spherical, oval, or discoidal in shape.

6. The nanoparticle delivery vehicle of claim 1, wherein the nanoparticle delivery vehicle has a molecular weight of approximately 120,000 to 500,000 Dalton.

7. The nanoparticle delivery vehicle of claim 1, wherein the nanoparticle delivery vehicle has approximately the same electrophoretic mobility, size, and chemical composition as native high density lipoproteins.

8. The nanoparticle delivery vehicle of claim 1, wherein the nanoparticle delivery vehicle further comprises a pharmaceutical agent.

9. The nanoparticle delivery vehicle of claim 8, wherein the pharmaceutical agent is located in the core of the nanoparticle delivery vehicle.

10. The nanoparticle delivery vehicle of claim 8, wherein the pharmaceutical agent is paclitaxel, or a lipophilic drug.

11. The nanoparticle delivery vehicle of claim 8, wherein the pharmaceutical agent is valrubicin.

12. The nanoparticle delivery vehicle of claim 1, wherein the core of the nanoparticle represents a lipophilic, nonaqueous environment.

13. The nanoparticle delivery vehicle of claim 1, wherein the lipid binding protein component is an apolipoprotein.

14. The nanoparticle delivery vehicle of claim 1, wherein the lipid binding protein component is apolipoprotein A-I or an analog thereof.

15. The nanoparticle delivery vehicle of claim 1, wherein the lipid binding protein component is modified to enhance the targeting efficacy of the drug.

16. The nanoparticle delivery vehicle of claim 1, wherein the lipid binding protein component is modified by the attachment of folic acid residues, antibodies or other ligands that target the surface of malignant cells and tumors.

17. The nanoparticle delivery vehicle of claim 1, further comprising a functional moiety which augments the efficacy of the pharmaceutical agent.

18. The nanoparticle delivery vehicle of claim 17, wherein the functional moiety targets the nanoparticle delivery vehicle via interaction with a cell surface receptor.

19. A method for delivering a drug of interest to a subject, comprising:
administering the nanoparticle delivery vehicle of claim 8 to the subject;
wherein the pharmaceutical agent is the drug of interest.

20. The method of claim 20, wherein the nanoparticle delivery vehicle is delivered parenterally, intravenously, intramuscularly, subcutaneously, transmucosally, or transdermally.

21. The method of claim 20, wherein the nanoparticle delivery vehicle is delivered parenterally.

22. A method of treating cancer in a subject, comprising:
administering the nanoparticle delivery vehicle of claim 8 to the subject;
wherein the pharmaceutical agent is a chemotherapeutic agent.

23. The method of claim 23, wherein the cancer is prostate cancer or ovarian cancer.

24. The method of claim 23, wherein the cancer is breast cancer.

25. A method for producing a self-assembly nanoparticle delivery vehicle, comprising the steps of:
a) mixing a phosphatidylcholine with a cholesterol and a cholesterol ester in dimethylsulfoxide to give a lipid-cholesterol mixture;
b) adding a pharmaceutical agent to the lipid-cholesterol mixture to give a lipid-cholesterol-drug mixture;
c) drying the lipid-cholesterol-drug mixture under nitrogen to give a thin film;
d) dispersing the thin film in about 3% dimethylsulfoxide to give a dispersed mixture;
e) subsequently mixing the dispersed mixture in a buffer to give a buffered mixture;
f) adding sodium cholate to the buffered mixture to give a sodium cholate mixture;
g) adding a lipid binding protein to the sodium cholate mixture to give a lipid-cholesterol-drug-lbp mixture;
h) incubating the lipid-cholesterol-drug-lbp to give an incubated mixture; and
i) subjecting the incubated mixture to dialysis with multiple buffer changes to facilitate the self assembly of the nanoparticle delivery vehicle.

26. The method of claim 26, wherein the phosphatidylcholine is egg yolk phosphatidylcholine.

27. The method of claim 27, wherein the ratio of egg yolk phosphatidylcholine:cholesterol:cholesterol oleate is approximately 3.8:1:88.5.

28. The method of claim 26, wherein the lipid binding protein is apolipoprotein A-I.
